(19) United States
(12) Patent Application Publication
(10) Pub. No.: US 2021/0259871 A1
(43) Pub. Date: Aug. 26, 2021

(54) DYNAMIC ANKLE ORTHOSIS DEVICES, SYSTEMS, AND METHODS

(71) Applicant: University of Tennessee Research Foundation, Knoxville, TN (US)

(72) Inventors: Denis J. DiAngelo, Germantown, TN (US); Chloe Chung, Lakeland, TN (US); Michael Parker, Memphis, TN (US)

(21) Appl. No.: 16/497,329

(22) PCT Filed: Apr. 5, 2018
(86) PCT No.: PCT/US18/26254

§ 371 Date: Sep. 24, 2019

Related U.S. Application Data
(60) Provisional application No. 62/481,741, filed on Apr. 5, 2017.

(51) Int. Cl. A61F 5/01

(52) U.S. Cl. CPC A61F 5/0127; A61F 2005/0165

(57) ABSTRACT

The present subject matter relates to orthotic devices, systems, and methods configured to support an ankle joint. In such devices, systems, and methods, a calf sleeve is configured to be secured about a leg of a user, a foot plate is configured to be secured about a foot of the user, and a distractive force mechanism is connected between the calf sleeve and the foot plate. The distractive force mechanism is configured to generate a force between the foot plate and the calf sleeve acting bidirectionally across the ankle to substantially offload bodyweight of the user passing through the ankle and lower limb.

### RELATED APPLICATIONS

The presently disclosed subject matter claims priority to and the benefit of U.S. Provisional Patent Application Ser. No. 62/481,741, filed Apr. 5, 2017; the disclosure of which is incorporated herein by reference in its entirety.

### TECHNICAL FIELD

The subject matter disclosed herein relates generally to orthotic devices. More particularly, the subject matter disclosed herein relates to orthotic devices, systems, and methods configured to support an ankle joint and/or lower leg.

### BACKGROUND

Three bones come together to form the ankle: the talus, tibia, and fibula. The talus connects the ankle to the foot while the tibia and fibula combine to form the lower leg. These three bones form the ankle mortise, a U-like shaped structure that allows for plantar and dorsiflexion, the movement of the foot in the sagittal plane. The ankle mortise comprises a medial and lateral malleolus, bony structures formed by the distal portions of the tibia and fibula respectively, generally indicating the end points of the rotational axis of the ankle. The rotational axis passing through the medial and lateral malleolus is not perpendicular to the sagittal plane. The hind foot is connected to the ankle and comprises 4 bones: the talus, calcaneus, cuboid, and navicular. These four bones combine to form the subtalar joint, calcaneocuboid joint, and talonavicular joint. The subtalar joint allows for inversion and eversion of the foot.

Common injuries of the ankle include fractures and sprains, particularly for those affected by degenerative pathologies such as arthritis (both rheumatoid and osteo) or diabetes. Both of these patient groups commonly use bracing as a non-operative treatment technique. Furthermore, bracing can likewise be helpful in the treatment of other lower leg injuries (e.g., tibial stress fractures).

Depending on the type and severity of the pathology, multiple different kinds of braces can be used as treatment options. Three kinds of braces currently used are stabilizing braces, energy storing braces, and patellar tendon bearing braces. Stabilizing braces reduce ankle and foot motion in one or more planes of motion. The reduction of this motion is said to decrease inflammation and might provide some pain relief. In some configurations, stabilizing rigid braces allow no articulation at the ankle joint, thus restricting motion in both the sagittal and coronal planes. Examples of these include rigid ankle foot orthosis (AFO) such that would be made by an orthotist via a mold made of the patient's lower leg. Another common example of a stabilizing rigid brace is the standard walking boot. These orthotics are primarily used when the patient has a degenerative ankle-hind foot disease (Kitaoka).

Stabilizing mobility braces restrict coronal motion, similar to the stabilizing rigid braces, but allow for motion in the sagittal plane (i.e., permitting plantar- and dorsi-flexion). People with arthritic ankles tend to be sensitive to motion in the coronal plane, so restricting such motion but allowing sagittal movement generates a more normal walking motion than achievable from a rigid brace. However, since the axis of rotation of the stabilizing mobility braces is perpendicular to the sagittal plane true physiological ankle movement is not permissible. These braces can be customized by an orthotist using polyethylene similar to the rigid brace or can be made with a design created by a separate company. Examples of such stabilizing mobility braces include a Richie Brace, a DonJoy Velocity ankle brace, and a leather and metal double upright AFO created by an orthotist.

Energy storing braces are used for patients with severe lower-extremity weakness. These braces take some of the load applied to the injured leg during activity and store it via deformation of a material, usually carbon fiber, which then provides a propulsion force when unloading the leg. This force acts to compensate for a lack of musculature and/or structure in the injured lower leg. Examples of such energy storage braces include an Intrepid dynamic exoskeletal orthosis, a PHAT brace, and a BlueROCKER brace.

Patella Tendon Bearing (PTB) ankle foot orthoses function as load sharing orthotics. Examples of patella tendon bearing braces include a full orthotic with shoe insert and a patella wrapping portion attached to a shoe, such as by a double-upright coupling structure. The logic behind the design is that the top/proximal portion of the brace which wraps around the calf and patella provides an alternate structure for the load to flow down. In this way, PTB orthoses provide passive load sharing by having the joint effectively undergo a physical height reduction. These braces must be highly nustomized with good fit to function properly. For example, where the axes of coupling to the shoe or shoe insert are not aligned with the anatomical flexion and/or extension of the foot, such bracing configurations can restrict ankle motion. Also, it is difficult to control exactly how much load sharing the PTB brace contributes.

### SUMMARY

In accordance with this disclosure, orthotic devices, systems, and methods configured to support an ankle joint are provided. In one aspect, a dynamic ankle orthosis system is provided in which a calf sleeve is configured to be secured about a leg of a user, a foot plate is configured to be secured about a foot of the user, and a distractive force mechanism is connected between the calf sleeve and the foot plate. In this configuration, the distractive force mechanism is configured to generate a force between the foot plate and the calf sleeve acting bidirectionally across the ankle to substantially offload bodyweight of the user passing through the ankle and lower limb.

In another aspect, a method for offloading at least a portion of a user's bodyweight at an ankle or lower leg of the user is provided. The method comprises securing a calf sleeve about a leg of the user, securing a foot plate about a foot of the user, connecting a distractive force mechanism between the calf sleeve and the foot plate, and generating a force by the distractive force mechanism between the foot plate and the calf sleeve acting bidirectionally across the ankle to substantially offload bodyweight of the user passing through the ankle and lower limb.

Although some of the aspects of the subject matter disclosed herein have been stated hereinabove, and which are achieved in whole or in part by the presently disclosed subject matter, other aspects will become evident as the description proceeds when taken in connection with the accompanying drawings as best described hereinbelow.

The features and advantages of the present subject matter will be more readily understood from the following detailed description which should be read in conjunction with the accompanying drawings that are given merely by way of explanatory and non-limiting example.

### BRIEF DESCRIPTION OF THE DRAWINGS

The features and advantages of the present subject matter will be more readily understood from the following detailed description which should be read in conjunction with the accompanying drawings that are given merely by way of explanatory and non-limiting example, and in which:

FIG. 1 is a perspective side view of a dynamic ankle orthosis system according to an embodiment of the presently disclosed subject matter.

FIGS. 2A and 2B are side views of a calf sleeve of a dynamic ankle orthosis system according to two embodiments of the presently disclosed subject matter.

FIG. 3 is a front view of an anterior sleeve portion of a calf sleeve of a dynamic ankle orthosis system according to an embodiment of the presently disclosed subject matter.

FIG. 4 is a top view of a calf sleeve of a dynamic ankle orthosis system according to an embodiment of the presently disclosed subject matter.

FIGS. 5A and 5B are front views of an anterior sleeve portion of a calf sleeve of a dynamic ankle orthosis system according to two embodiments of the presently disclosed subject matter.

FIG. 5C is a side view of a calf sleeve of a dynamic ankle orthosis system according to an embodiment of the presently disclosed subject matter.

FIGS. 6A and 6B are side views of elements of a distractive force mechanism of a dynamic ankle orthosis system according to an embodiment of the presently disclosed subject matter.

FIGS. 7A and 7B are front and side views of a distractive force mechanism of a dynamic ankle orthosis system according to an embodiment of the presently disclosed subject matter.

FIGS. 8A and 8B are front and side views of a distractive force mechanism of a dynamic ankle orthosis system according to an embodiment of the presently disclosed subject matter.

FIGS. 9A and 9B are perspective side views of a foot plate of a dynamic ankle orthosis system according to two embodiments of the presently disclosed subject matter.

FIGS. 10A and 10B are front and side views of a dynamic ankle orthosis system including an alternative configuration for a foot plate according to an embodiment of the presently disclosed subject matter.

FIG. 11 is a front view of a user's foot secured within a foot plate of a dynamic ankle orthosis system according to an embodiment of the presently disclosed subject matter.

FIGS. 12A through 12C are side views of arrangements of a foot plate of a dynamic ankle orthosis system according to embodiments of the presently disclosed subject matter.

FIGS. 13A and 13B are side and rear views of a dynamic ankle orthosis system according to an embodiment of the presently disclosed subject matter.

FIGS. 14A and 14B are side and front views of a dynamic ankle orthosis system according to an embodiment of the presently disclosed subject matter.

### DETAILED DESCRIPTION

The present subject matter provides dynamic ankle orthosis devices, systems, and methods. A primary goal of dynamic ankle orthosis is to offload at least a portion of the force encountered by the lower leg, ankle joint, and parts of the foot in stance/gait without introducing excessive off-axis forces at the ankle's rotational axis and resistance to ankle motion. In some embodiments, such offloading is provided by a distractive force (i.e., a force acting in the opposite direction of the body's weight) applied between the foot and the lower leg. The primary advantage of using a distractive force is the ability to control how much offloading occurs. Introducing a new force to the system creates a new design problem: if the forces are not passed onto the brace approximately at the same location as the ankle's rotational axis, new off-axis forces will be generated by the brace which will affect the user's ability to walk normally.

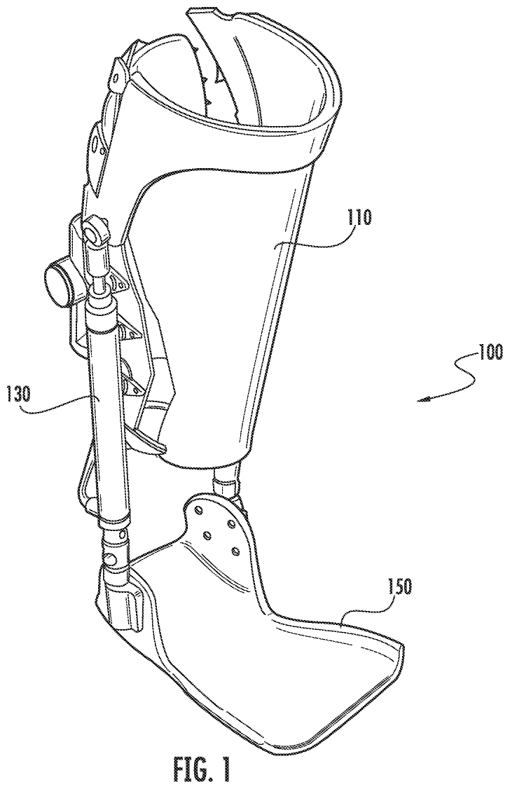

Accordingly, in one aspect, the present subject matter provides a dynamic ankle orthosis system. In one embodiment illustrated in FIG. 1, a dynamic ankle orthosis system, generally designated 100, includes a socket cuff/calf sleeve 110 that is configured to fit securely around the user's calf portion of the lower leg, a distractive force mechanism 130 coupled to calf sleeve 110, and a foot plate 150 coupled to distractive force mechanism 130 and allows for the transfer of the distractive force, which allows partial body weight loads to bypass the lower leg, ankle joint, and parts of the foot.

In some embodiments, calf sleeve 110 of dynamic ankle orthosis system 100 has a structure that is analogous in some ways to the design of the upper portion of conventional rigid ankle foot orthoses. Those having skill in the art should recognize, however, that any of a variety of further configurations for calf sleeve are provided by the presently-disclosed subject matter, including configurations using multiple different materials and techniques. For example, dynamic ankle orthosis system 100 can incorporate any of a variety of different calf sleeve configurations and/or methods, including but not limited to air bladders, suspension material (e.g., hook-and-loop fasteners, belt-like attachment, shoe laces), leather/shoe laces (i.e., a boot-style cuff), or a hard shell with padding (e.g., custom made with casting).

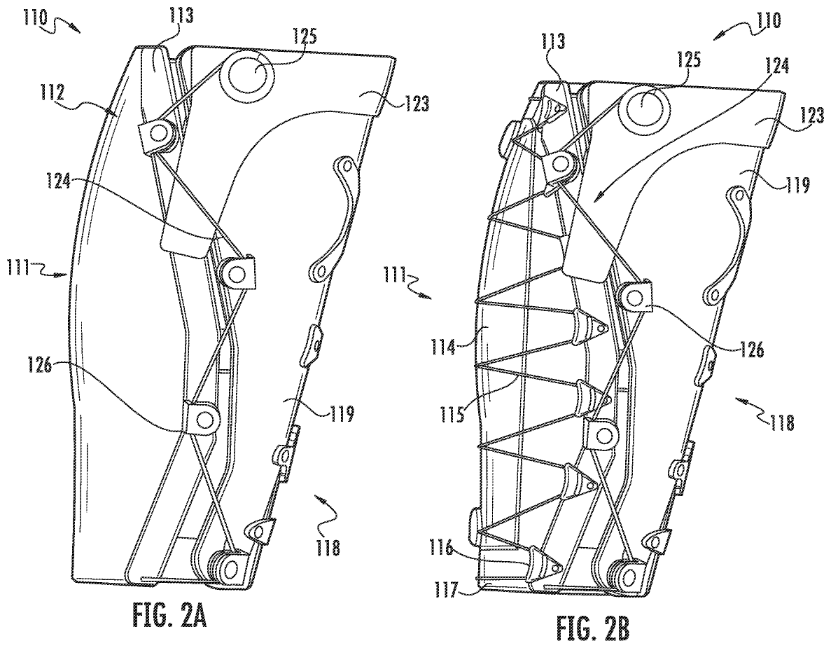

In any configuration, calf sleeve 110 provides a secure fit on the user's leg to ensure that minimal distractive force is lost as a result of the calf sleeve slipping. In this regard, in some embodiments, such as those illustrated in FIGS. 2A-6C, the calf sleeve 110 is designed to engage the lower leg by wrapping around the calf and shin and by conforming to their natural anatomical profiles. In the illustrated embodiments, this engagement is achieved generally by coupling together a posterior sleeve portion 111 and an anterior sleeve portion 118 about the lower leg of the user. Various different configurations for each of posterior sleeve portion 111 and anterior sleeve portion 118 can be used in a variety of combinations to achieve a desired fit of dynamic ankle orthosis system 100 on a given user.

In some embodiments, such as is shown in FIG. 2A, posterior sleeve portion 111 includes a posterior engagement material 112 that is configured to contain the muscle belly of the calf of the user. In some embodiments, posterior engagement material 112 includes a textile mesh that spans between two elongated posterior fixation members 113, which can be narrow, semi-rigid structures that are configured to be positioned on either side of the user's calf.

Alternatively, as illustrated in FIG. 2B, posterior sleeve portion 111 includes a posterior engagement bladder/padding 114 in place of posterior engagement material 112. Posterior engagement padding 114 can be held in place with an organization of one or more cables or laces 115, which can be routed across posterior engagement padding 114 one or more times to secure it against the user's calf. In some embodiments, the laces 115 can loop through lace guides 116 to control the position of laces 115 across posterior engagement padding 114 and maintain substantially consistent tension across the user's calf. In any configuration, a compression sleeve 117 can be worn over the lower leg and beneath calf sleeve 110, such as beneath posterior engagement padding 114, to improve comfort of calf sleeve 110.

The selection of the particular configuration for posterior sleeve portion 111 of calf sleeve 110 can be made based on any of a variety of design considerations, including comfort of the wearer and experience with past designs. In addition, certain embodiments of posterior sleeve portion 111 may be better suited for a given wearer's anatomy to securely engage the wearer's leg without limiting or unnecessarily constraining the wearer's calf muscle belly.

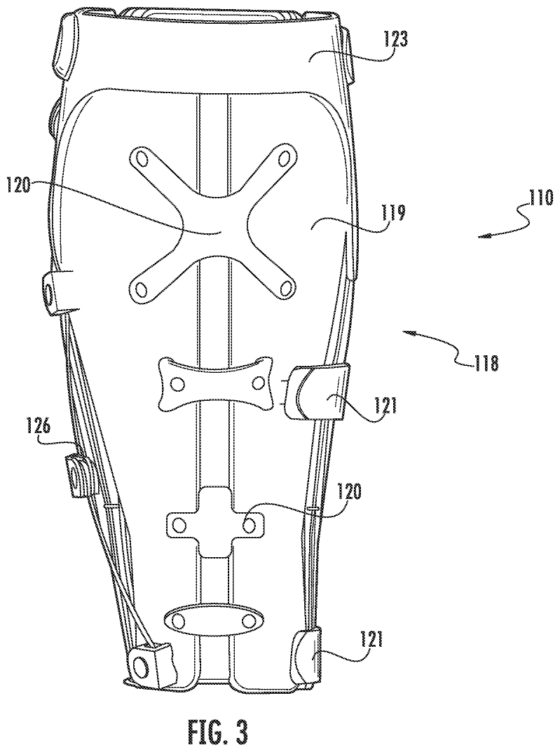

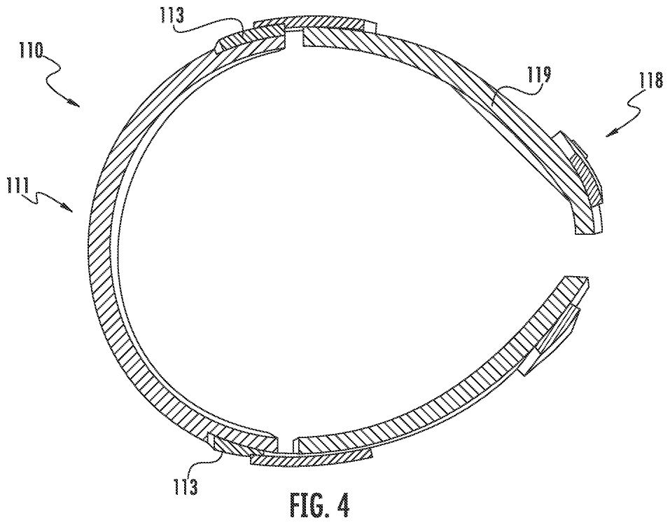

In any configuration, calf sleeve 110 further includes an anterior sleeve portion 118 that is configured to engage the shin of the user, such as by running along the flat portion of the tibial shaft. In one embodiment illustrated in FIGS. 3 and 4, one or more anterior engagement blades 119 are configured to mate with the natural anatomical profile of the bony shin region of the user. In the illustrated embodiment, anterior engagement blades 119 include two semi-rigid members that are configured to contact the shin of the user. In some embodiments, one or more connectors 120, which may vary in geometry, are used to hold anterior engagement blades 119 in a desired relative position. In this arrangement, the use of one or more anterior engagement blades 119 can be modular and can thus can be readily adapted without custom molding to provide a sufficiently secure fit for users having different leg shapes.

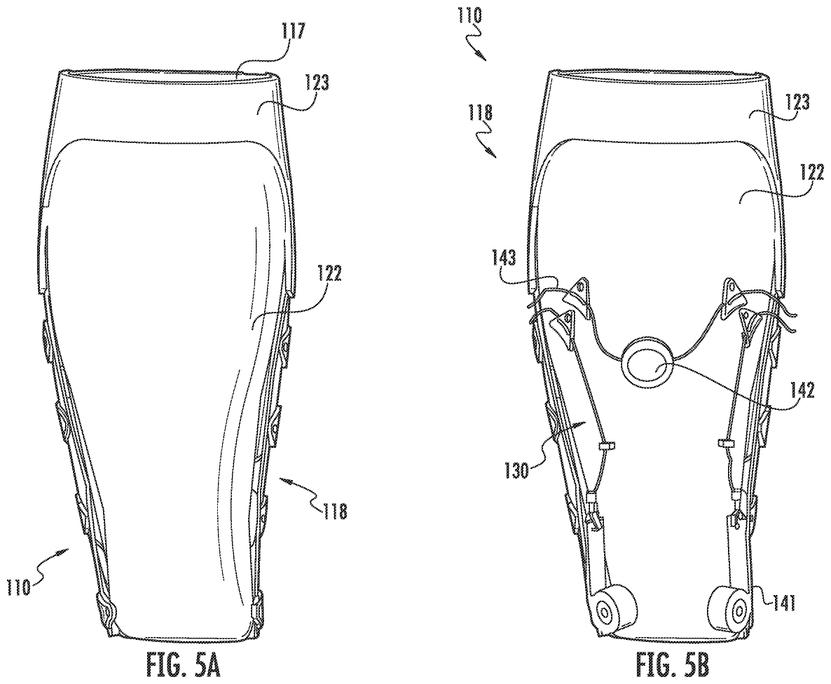

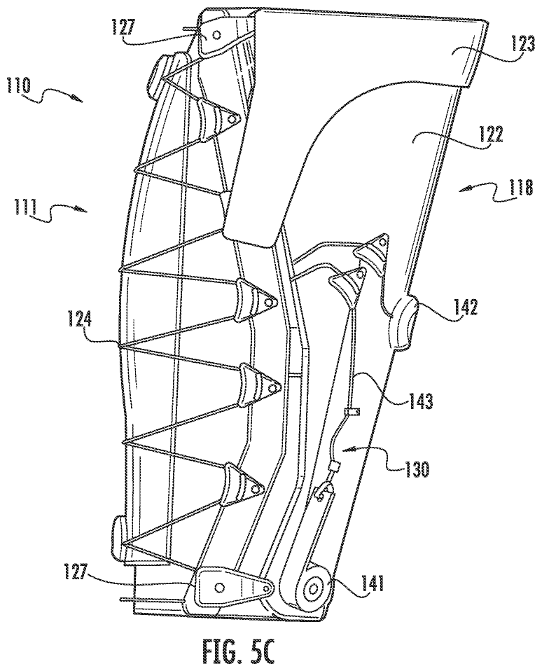

Alternatively, in another embodiment of the calf sleeve is illustrated in FIGS. 5A-5C, anterior sleeve portion 118 includes a unitary anterior engagement blade 122. In some embodiments, unitary anterior engagement blade 122 is custom designed and fit to a given user's anatomy, which can provide a more stable engagement of the user's shin. In addition, in some embodiments, elements of distractive force mechanism 130 can be mounted to unitary anterior engagement blade 122 as illustrated in FIGS. 5B and 5C.

Again, the selection of the particular configuration for anterior sleeve portion 118 of calf sleeve 110 can be made based on any of a variety of design considerations, including personal preference and comfort of the wearer. In addition, certain embodiments of anterior sleeve portion 118 may better provide a geometric fit with a given wearer's anatomy to securely engage the bone surface of the wearer's tibia.

Regardless of the particular configuration of each of the elements of calf sleeve 110, in some embodiments, posterior sleeve portion 111 (e.g., posterior engagement material 112 or posterior engagement padding 114) and anterior sleeve portion 118 (e.g., anterior engagement blades 119 or unitary anterior engagement blade 122) are held together by one or more coupling elements to maintain a secure fit about the user's lower leg. In the embodiments illustrated in FIGS. 2A-3, a cable tensioning system is used for this coupling. In this configuration, an arrangement of one or more cables 124 and pulleys 126 spans the gap between posterior sleeve portion 111 and anterior sleeve portion 118 of calf sleeve 110 to help tighten the calf sleeve 110 as a whole around the lower leg. In some embodiments, to maintain tension in this coupling arrangement, cables 124 are connected to a tension control unit 125, such as is shown in FIGS. 2A and 2B. As illustrated in FIGS. 2A-3, in some embodiments, such a system can be provided on only one side of calf sleeve 110, while anterior sleeve portion 111 and posterior sleeve portion 118 on the opposite side of calf sleeve are coupled together using an alternative fastener, such as one or more straps 121 (e.g., using hook-and-loop fasteners). Alternatively or in addition, anterior sleeve portion 111 and posterior sleeve portion 118 of calf sleeve 110 can also be connected by clip connectors 127 as shown in FIG. 5C, or other coupling elements (e.g., simple laces, hook-and-loop straps, or ratchet straps) known to those having ordinary skill in the art can be used for this purpose.

In any configuration of calf sleeve 110, a calf ring 123 can be fixed to or embedded within the material of the caudal portion of anterior sleeve portion 118 (e.g., to or within anterior engagement blade 119 or unitary anterior engagement blade 122). Calf ring 123 serves as a rigid point of fixation for distractive force mechanism 130. In addition, in some embodiments, calf ring 123 further helps to provide structural stability of calf sleeve 110 and provides an anchor point for arrangements of laces 115 and/or cables 124 that are used to maintain tension of the elements of calf sleeve 110 about the user's leg as discussed above. In some embodiments, calf ring 123 is made of metal, carbon fiber, or dense polymer. Those having ordinary skill in the art will recognize, however, that calf ring 123 as shown and described is optional and not an essential component of the device, since it only serves to provide additional structure for cases when the brace needs reinforcement. Alternatively, the reinforcement can come from an embedded bracket within the material of calf sleeve 110 or a thickening of material of calf sleeve 110 at a desired location.

As discussed above, distractive force mechanism 130 is connected to calf sleeve 110, such as at calf ring 123, and is configured to introduce a distractive force to dynamic ankle orthosis system 100. Similarly to the way in which a variety of configurations and combinations of elements can be used to provide secure fixation for calf sleeve 110, multiple different configurations and combinations of elements for distractive force mechanism 130 can be used to provide this force. In some embodiments, for example, dynamic ankle orthosis system 100 uses one or more pneumatic cylinders to create the distractive force. In some embodiments, these cylinders are attached on the lateral and medial sides of dynamic ankle orthosis system 100 and act in series via pneumatic components that are connected via tubing. That being said, those having ordinary skill in the art will recognize that various different pneumatic cylinders can be used to allow for more or less stroke length or greater force generation at lower pressures (requires larger bore).

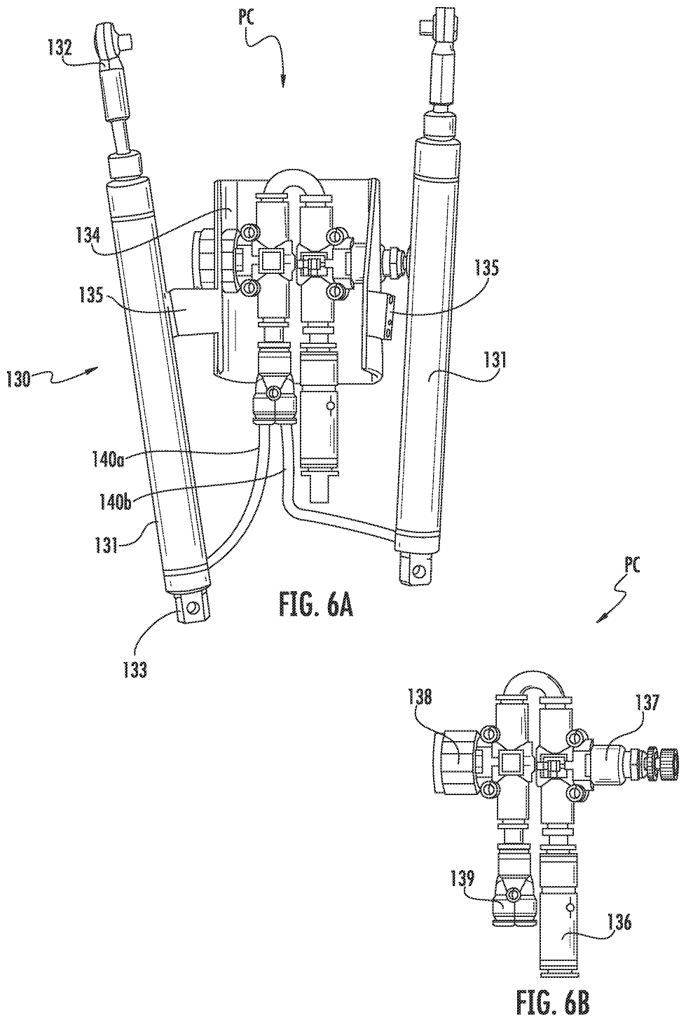

FIGS. 6A-6B illustrate a configuration in which pneumatic components are used to create the distractive force for dynamic ankle orthosis system 100. In such an embodiment, distractive force mechanism 130 comprises pneumatic cylinders 131 that attach proximally to calf sleeve 110. In the embodiment illustrated in FIG. 6A, for example, each of pneumatic cylinders 131 includes an upper connector 132 that is configured for connection to calf sleeve 110, such as at calf ring 123. In the embodiment illustrated in FIG. 6A, each upper connector 132 is a tie rod end, although those having ordinary skill in the art will recognize that other attachment mechanisms (e.g. ball joints) can be used to achieve the desired load connection between calf sleeve 110 and distractive force mechanism 130. Likewise, pneumatic cylinders 131 are further configured to attach distally to foot plate 150 at the bottom of each cylinder 131, such as via a lower connector 133, which can be a tie rod, a ball joint, or any of a variety of other connector types that can be configured to be coupled with a bracket described below.

In some embodiments, a pressure control assembly PC in communication with pneumatic cylinders 131 can be housed within a pouch 134, which in some embodiments is located on the posterior surface of calf sleeve 110. Pouch 134 attaches to the calf sleeve 110 using one or more fasteners, such as one or more straps 135 (e.g., hook-and-loop straps). In some embodiments, pressure control assembly PC includes pressure control features that are operable to maintain a selected pressure within pneumatic cylinders 131. In the illustrated embodiment, for example, a check valve 136 is configured to insert air into the system. This check valve 136 allows air flow in only one direction so any air introduced into the system will not escape via its entrance route. This arrangement allows pneumatic cylinders 131 to maintain pressures and consistently produce an outward force. In-line with check valve 136, a slow release valve 137 allows the user to slowly release pressure from the pneumatic system if desired. In some embodiments, for example, slow release valve 137 is configured such that each time it is used, the valve will release an incremental amount of pressure (e.g., 10 PSI) from the system. Distractive force mechanism 130 in this embodiment can further include an air pressure gauge 138 that allows the user to see the amount of air pressure (e.g., measured in PSI or Bar) currently in the pneumatic system. This feedback can help the user control the amount of distractive force dynamic ankle orthosis system 100 is currently providing.

Distractive force mechanism 130 can further include a split valve 139 in communication between pressure control assembly PC and pneumatic cylinders 131 that splits the primary tubing (e.g., ¼ inch) into multiple tubes (e.g., ⅛-inch) that then travel to the corresponding one of pneumatic cylinders 131 and provide them with air. In the illustrated embodiment, split valve 139 is attached to one of pneumatic cylinders 131 by a first tube 140a (e.g., a lateral tube) and to the other of pneumatic cylinders 131 by a second tube 140b (e.g., a medial tube).

In this illustrated embodiment, pneumatic cylinders 131 contain a four-inch stroke length and provide a force of 25 lbs. for every 100 PSI of air pressure inserted. In this configuration, since pneumatic cylinders 131 are in series, if one inserts 100 PSI of air pressure, pneumatic cylinders 131 will provide a total of 50 lbs. of force. Pressure control assembly PC serves to control the action of pneumatic cylinders 131.

Regardless of the particular elements of distractive force mechanism 130, pneumatic cylinders 131 can be configured to achieve any of a variety of different load responses. Pneumatic cylinders 131 can also be configured to allow different ankle mobility conditions during these active loading states. For instance, in some embodiments, the bottom ends of both of pneumatic cylinders 131 are connected in a substantially closed system as shown in FIG. 6A so that if one of them extends the other one will shorten, thereby allowing unconstrained inversion/eversion ankle motion. Alternatively, in other embodiments, one air tube can be connected to the top of one cylinder, and the other air tube can be connected to the bottom of the other cylinder. In such an arrangement, distractive force mechanism 130 will be loaded but will not be allowed to lengthen or shorten, thus essentially acting as a rigid stabilizing joint.

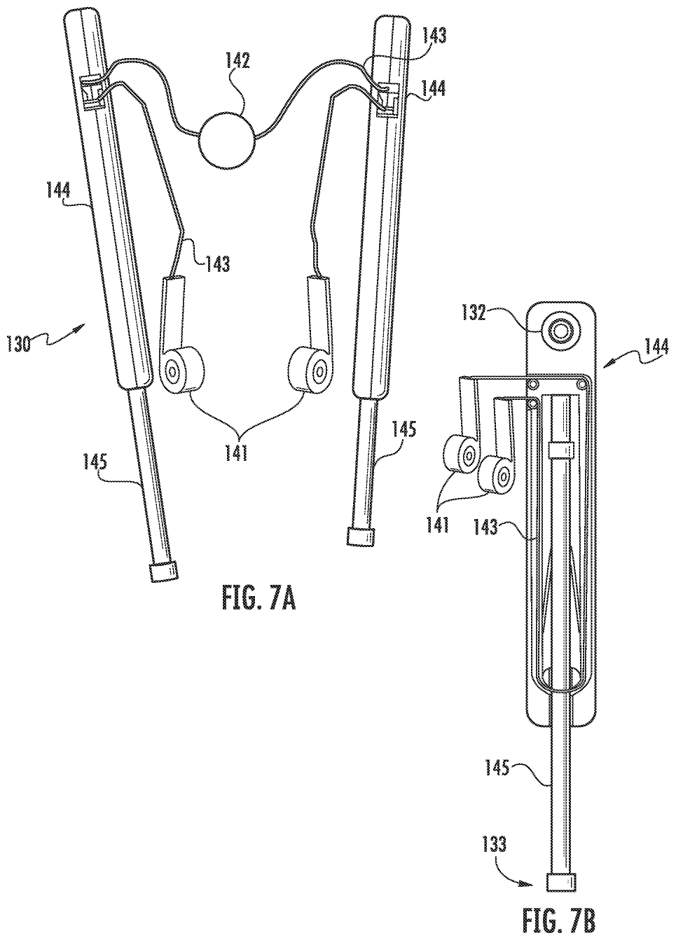

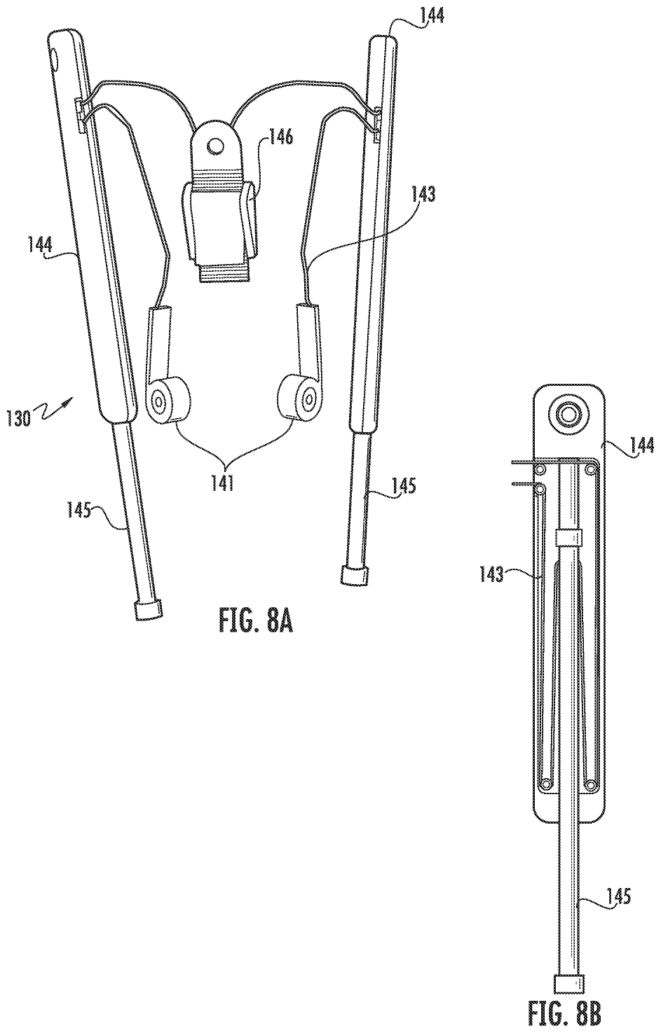

Alternatively, FIGS. 7A through 8B illustrate embodiments in which distractive force mechanism 130 is implemented using a mechanical action distractive force mechanism that uses one or more constant force springs 141. Unlike conical springs that have constant rate of force per unit length of spring deformation, a constant force spring exerts a specific force through the entire range of motion of the spring. In some embodiments, springs 141 are laminated or stacked to provide an increased force output without substantially changing the size. In some embodiments, springs 141 are held in housings, such as by pinned connectors and ball bearings or by being enclosed in a cavity.

In any form, these springs 141 can be attached on the sides of dynamic ankle orthosis system 100—medially and laterally—or on one of the anterior or posterior portion. In some configurations, springs 141, as illustrated in FIGS. 5B and 5C, can be attached to an anterior sleeve portion 118 of calf sleeve 110. Although one arrangement for springs 141 on calf sleeve 110 is shown, those having ordinary skill in the art will recognize that similar functionality can be achieved with springs 141 positioned in any of a variety of positions, such as in a medial configuration in which springs 141 are positioned and enclosed on the center of anterior sleeve portion 118, in a lateral configuration in which springs 141 are positioned and enclosed on outer edges of anterior sleeve portion 118, or in a modular configuration in which springs 141 are enclosed in independent housings that are locatable anywhere on the assembly.

In any arrangement, a spring-based configuration for distractive force mechanism 130 can comprise one or more constant force springs 141 and a tension control unit 142, which can vary in design. In some embodiments, constant force springs 141 function to generate a distractive force by way of a cable tension system that acts between calf sleeve 110 and foot plate 150. In the embodiments illustrated in FIGS. 7A through 8B, for example, a tension cable 143 connected to each of constant force springs 141) is routed through a housing 144 that is coupled to calf sleeve 110. Any of a variety of cable routing configurations can be used, two examples of which are illustrated in FIGS. 7B and 8B. In these configurations, tension cable 143 is routed through housing 144 over pulleys. A rod 145 that is coupled to foot plate 150 is configured to translate in a path within housing 144, and tension cable 143 is connected to a portion of rod 145 such that rod 145 is displaced downward when tension is applied to tension cable 143. Similarly to the configuration of pneumatic cylinders 131, in some embodiments, housing 144 includes an upper connector 132 that is configured for connection to calf sleeve 110, such as at calf ring 123. In some embodiments, upper connector 132 is a tie rod end or swivel ball bearing. Likewise, rod 145 further includes a lower connector 133 that is configured to attach distally to foot plate 150 at the bottom of rod 145, such as using a threaded connection, another tie rod end, a ball joint, or with a bracket.

Tension within tension cable 143 can be controlled using a standard tension control unit 142 as shown in FIG. 7A, using a ratchet bracket 146 as shown in FIG. 8A, or using any of a variety of other tension control mechanisms known in the art. In any configuration, the constant force-displacement relationship provides a benefit compared to existing bracing configurations by enabling an active off-loading of the joint, and thus the joint does not have to reduce in height to experience a load change.

Furthermore, although some particular structures of dynamic ankle orthosis system 100 are described with respect to various embodiments that uses either one or more pneumatic cylinders 131 or one or more constant force springs 141 in distractive force mechanism 130, any of a variety of alternative configurations for distractive force mechanism 130 are also contemplated by the presently disclosed subject matter. In some embodiments, for example, air baffles or air bladders can be used as the distractive force mechanism. Similarly to pneumatic cylinders, as the pressure increases inside of such elements, the amount of force exerted can increase. In contrast to pneumatics, however, the attachment point for such air bladders can be designed such that they are attached at calf sleeve 110 and the bladders can contribute to both lower leg attachment and distractive force. The mechanism by which the bladders are inflated can be an external device similar to what is currently used (e.g., a bladder-like nipple similar to those used in some shoe wear (e.g. Reebok™ Pumps)) or a device that is placed in the sole area of the shoe and with each step it activates the system and sends air to the system (i.e., an accumulator). In some embodiments, the system can include features that ensure the bladders are not over inflated (e.g., controlled via a bleeder valve), so once a certain pressure is achieved, air will just flow out into the environment.

In yet a further alternative, a physical displacement device (e.g., a ratcheting device) is provided as the distractive force mechanism, wherein two rods that overlap and can be ratcheted to increase rod length. If there is good attachment between calf sleeve and leg, the increase in rod length will engage soft tissue and provide a distractive force.

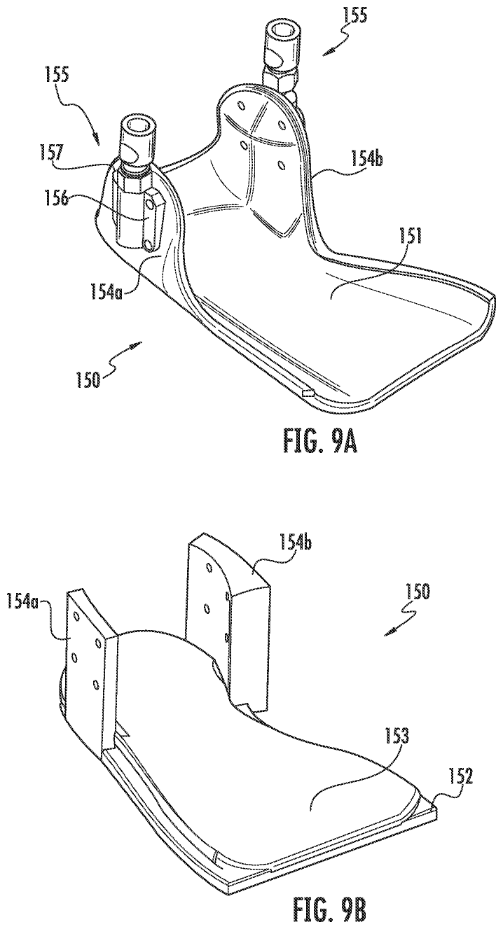

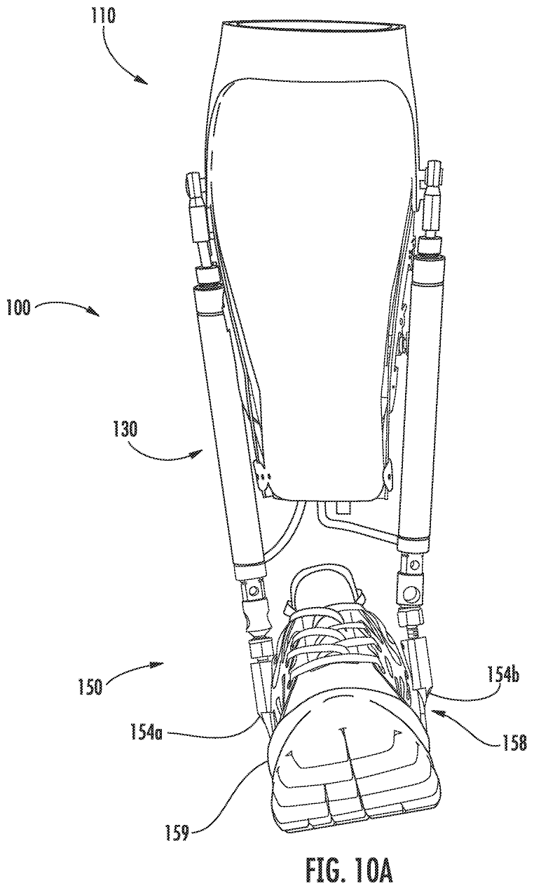

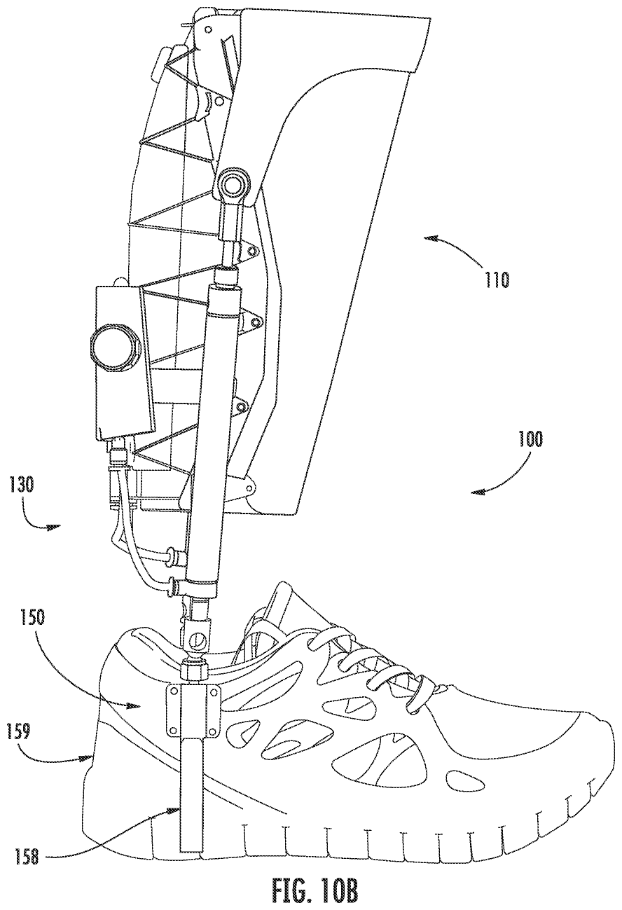

As discussed above, the foot plate 150 of dynamic ankle orthosis system 100 is coupled to distractive force mechanism 130 and thereby allows for the transfer of the distractive force to bypass the ankle joint and parts of the foot. In some embodiments, the design and build of foot plate 150 can be similar to existing fabrication techniques of ankle and heel cups used by orthotists. FIGS. 9A and 9B illustrate various implementations of foot plate 150 that are each designed to engage the foot and ankle complex. In some embodiments, such as that illustrated in FIG. 9A, a custom foot plate 151 comprises a moldable piece that wraps around the heel and extends beneath the foot to a specified length within a standard shoe. Alternatively, as illustrated in FIG. 9B, foot plate 150 can instead include a generic foot plate 152, which can be used with a foam insole 153 or other cushioning structure that is configured to conform to the user's foot. In any configuration, foot plate 150 can be either permanently installed or removably insertable into the sole portion of a standard shoe. Alternatively, in yet another embodiment illustrated in FIGS. 10A and 10B, foot plate 150 is implemented as a solid stirrup plate 158 that is integrated with or otherwise installed in a shoe 159 of the user. In any configuration, a lateral tab 154a and medial tab 154b extend from the insole portion of foot plate 150 near the ankle for connection to distractive force mechanism 130.

In contrast to conventional configurations, foot plate 150 according to the present subject matter allows for improved mobility. In this regard, in some embodiments, foot plate 150 includes a foot connector 155 that is configured to allow for full sagittal mobility with some mobility laterally and medially. In some embodiments, for example, foot connector 155 is mounted to each of the medial and lateral sides of foot plate 150, such as lateral tab 154a and medial tab 154b , such as is shown in FIG. 9A. Foot connector 155 is further configured for attachment to the corresponding component of distractive force mechanism 130, such as lower connectors 133 of pneumatic cylinders 131 or of rods 145, such as with a ball joint end. Although the particular configuration is shown and described with respect to the embodiment of FIG. 9A, those having ordinary skill in the art should recognize that such connection systems can likewise be applied to other configurations for foot plate 150, including those illustrated in FIGS. 9B, 10A, and 10B.

In some embodiments, foot plate 150 further includes an ankle adapter plate 156 that is either placed by the orthotist at the rotational axis 160 of the ankle or is modular and can be adjusted to be placed at or near bony landmarks (e.g., the malleoli) of the user as illustrated in FIG. 10. A ball joint 157 or other connector is attached to adapter plate 156, such as by threading into adapter plate 156. In some embodiments, it can be advantageous that adapter plate 156 is fixed to foot plate 150 such that ball joint 157 substantially aligned with rotational axis 160 of the ankle. Such alignment can be achieved by particularly designing the position of ball joint 157 based on the user's anatomy or by providing an adjustable connection between ball joint 157 and adapter plate 156 so that the relative position of these elements with respect to rotational axis 160 can be tuned as needed. In this configuration, the positioning of ball joint 157 can allow for substantially unconstrained multi-axis ankle motion without increasing the resistance to motion. As a result, dynamic ankle orthosis system 100 can introduce the desired distractive force while still allowing for improved ankle mobility.

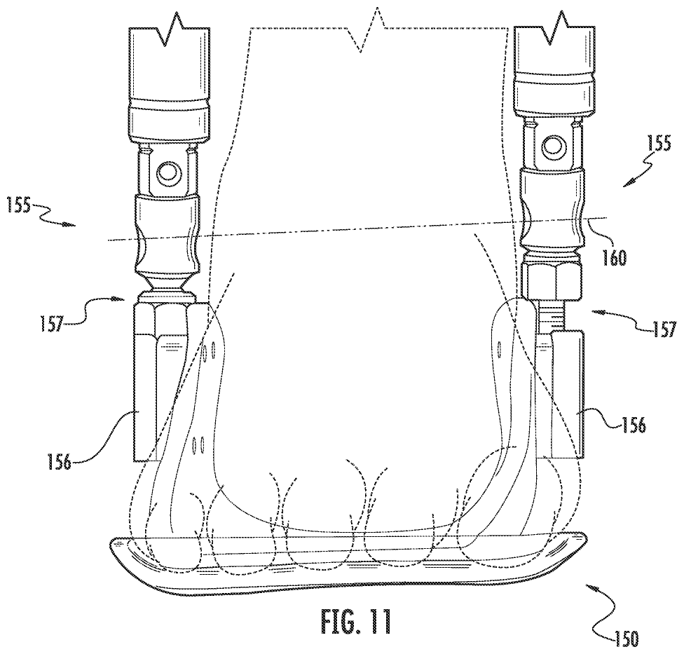

Alternatively, in some embodiments, the location of ball joints 157 can be intentionally offset caudal-cranially or anterior-posteriorly as illustrated in FIGS. 11A-11C. Specifically, FIG. 11A illustrates a posterior offset, FIG. 11B illustrates a neutral alignment, and FIG. 11C illustrates an anterior offset. Those having ordinary skill in the art will recognize, however, that additional configurations not illustrated can further be implemented by adjusting the positioning of lower connectors 133 with respect to the user's anatomy.

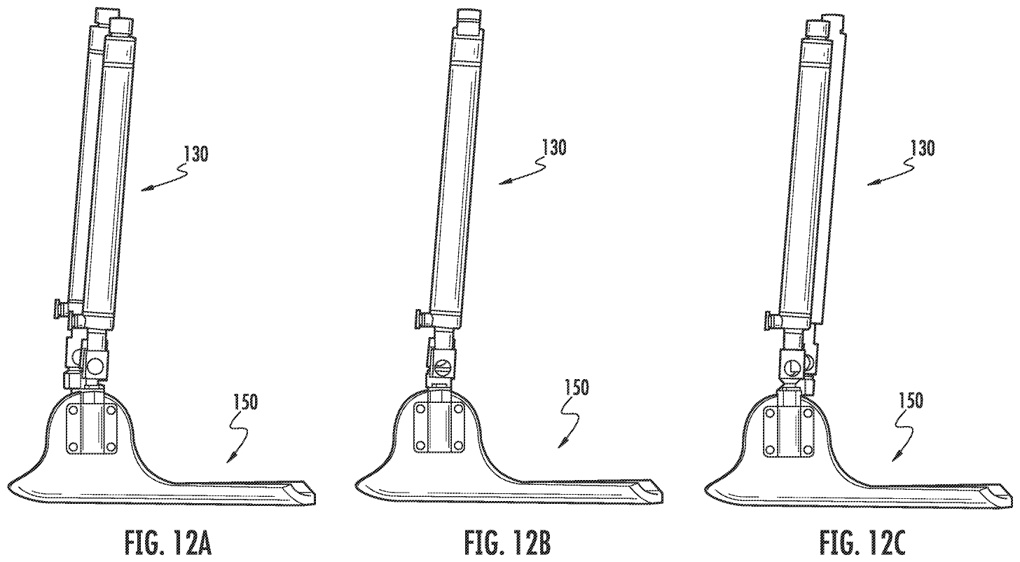

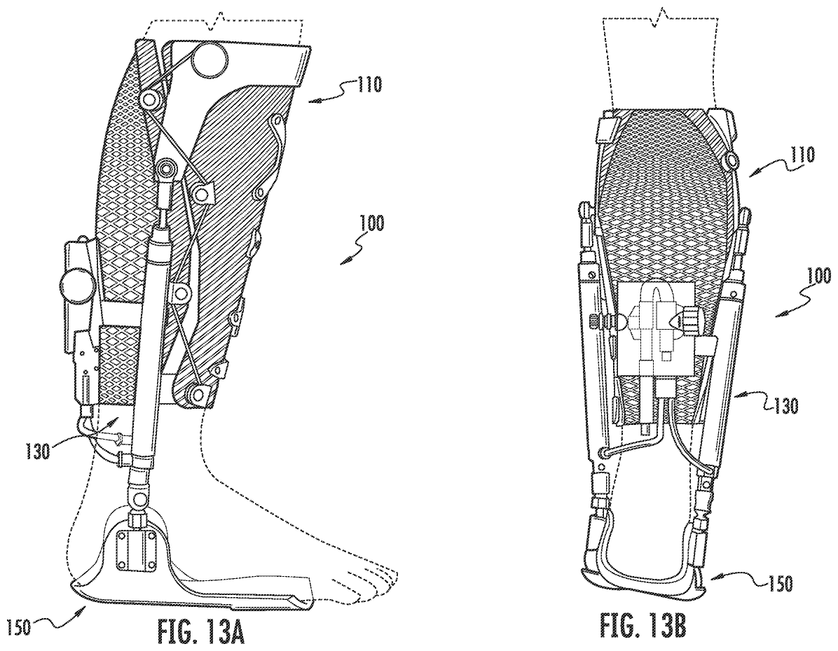

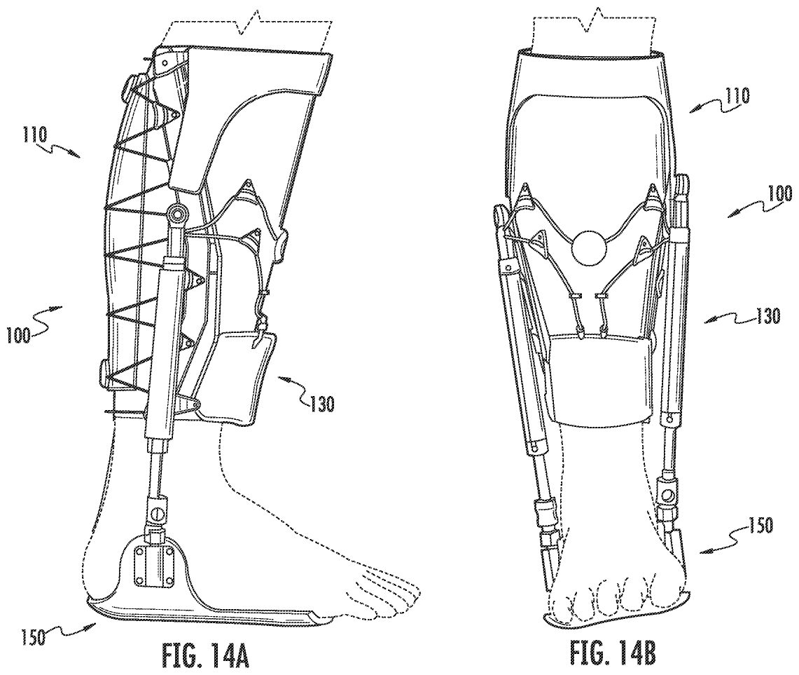

FIGS. 12A and 12B and FIGS. 13A and 13B illustrate embodiments of the complete, assembled dynamic ankle orthosis system 100. Although particular configurations for dynamic ankle orthosis system 100 are shown in these figures, the particular design and/or construction of each of calf sleeve, foot plate, and distractive force mechanism can be varied as discussed above in any of a variety of other combinations. In particular, for example, the attachment mechanism by which calf sleeve 110 is held in place can be varied to have any of a variety of forms, including a posterior sleeve portion 111 including one or more of a textile mesh (See, e.g., FIGS. 3A, 12A, or 12B) or a kind of cable-based tensioning system (See, e.g., FIGS. 3B, 5C, or 13A). Likewise, anterior sleeve portion 118 of calf sleeve 110 can be implemented in any of a variety of forms, including a plurality of anterior engagement partial blades 119 (See, e.g., FIGS. 2A-4) or a unitary anterior engagement blade 122 (See, e.g., FIGS. 5A-5C, 13A and 13B). With respect to distractive force mechanism 130, embodiments of dynamic ankle orthosis system 100 shown in FIGS. 6A and 6B implement distractive force mechanism 130 using pneumatic cylinders 131, whereas the embodiments shown in FIGS. 7A-8B implement distractive force mechanism 130 using a constant force spring 141 and cable tensioning system, which can be attached to anterior sleeve portion 118 of calf sleeve 110 as illustrated in FIGS. 5B-5C. With respect to foot plate 150, various configurations including a custom foot plate 151 shown in FIG. 9A, a generic foot plate 152 shown in FIG. 9B, or a solid stirrup plate 158 that is integrated with or otherwise installed in a shoe 159 of the user as shown in FIGS. 10A and 10B can be used.

Regardless of the particular combination of element configurations, however, dynamic ankle orthosis system 100 is operable to offload at least a portion of the force encountered by the lower leg, ankle joint, and parts of the foot in stance/gait without introducing excessive off-axis forces at the ankle's rotational axis and resistance to ankle motion. As discussed above, such offloading is achieved by the introduction of a distractive force between foot plate 150 and calf sleeve 110 acting bidirectionally across the ankle to substantially offload bodyweight of the user passing through the ankle and lower limb.

To test the effectiveness of the present dynamic ankle orthosis devices, systems, and methods to meet the design goals, three experiments were performed: a first experiment was conducted to verify the distractive force capabilities of the distractive force mechanisms, a second experiment was conducted to validate how much offloading the dynamic ankle orthosis provides at the ankle joint, and a third experiment was conducted to test the changes in mobility when the dynamic ankle orthosis is added to the leg. Summaries of these experiments are provided below.

### Experiment 1: Distractive Force Mechanism Validation

To validate the distractive force capacity and efficacy of dynamic ankle orthosis system 100, a first test of an embodiment of dynamic ankle orthosis system 100 using pneumatic cylinders 131 was performed to acquire force values to correspond with the pressures applied to the cylinders.

A plate was created with two clearance holes to place the 7/16-20 threaded ends of the cylinders through. A corresponding nut was then used to secure pneumatic cylinders 131 to the plate. They were each attached to a vertical fixture for positioning, which was securely attached to the platform of the robot, and placed beneath the upper load cell. The cylinders were connected in series in the same manner as when attached to dynamic ankle orthosis system 100. By connecting pneumatic cylinders 131 in series, equivalent pressures were delivered to the two cylinders, corresponding to the pressure value shown on the pressure gauge. (I.E., when the pressure gauge showed 40 PSI both cylinders were inflated to a pressure of 40 PSI.) Both cylinders acted on the load cell, so the reported values show the total amount of force delivered by both of the cylinders.

The compressive force on the load cell was acquired at various cylinder pressure values: 40, 50, 60, 70, and 80 PSI. Each pressure was tested four times and forces were exported and processed in Microsoft Excel. The pneumatic cylinders were emptied of their pressures between all runs and inflated to the targeted pressure to begin the new test. Table 1 shows the mean force values along with standard deviation for the force the pneumatic cylinders exerted when filled to certain pressures.

TABLE 1
Cylinder pressures with corresponding
mean force for two pneumatic cylinders

| | Dual Cylinders |
|---|---|
| Pressure (PSI) | Force (N) |
| 40 | 65.5 ± 2.5 |
| 50 | 87.2 ± 0.7 |
| 60 | 111.9 ± 2.2 |
| 70 | 135.5 ± 2.6 |
| 80 | 155.3 ± 1.3 |

### Experiment 2: Brace Force Assembly Testing

A second experiment was designed to quantify the offloading capabilities of dynamic ankle orthosis system 100 as a function of pneumatic pressure relative to body weight.

A testing fixture was created to measure the amount of load relief that the dynamic ankle orthosis provided. First and second load cells and were bolted to a bottom plate and wooden planks were then attached to the top of each of the first and second load cells and to give the user somewhere to stand. The user stood with their feet approximately shoulder width apart with one foot on the first load cell and the other foot on the second load cell. Vertical uprights were then attached to the bottom plate on the medial and lateral sides of the first load cell. The subject dons calf sleeve 110 of dynamic ankle orthosis system 100 with pneumatic cylinders 131 attached to both of calf sleeve 110 and the uprights. Sliding members in the uprights allowed for height adjustment of the attachment point for the bottom portion of pneumatic cylinders 131. In this configuration, brace force introduced by pneumatic cylinders 131 bypasses the first load cell and dissipates through the bottom plate.

Testing was performed in stance with each foot positioned on top of a single load cell. Calf sleeve 110 of dynamic ankle orthosis system 100 was placed on the right leg and the bottom portions of pneumatic cylinders 131 were secured into place approximately at the malleoli. A program recorded force and moment values over a specified time. For each test, the program was initiated and the user would stand for approximately five seconds without brace activation. Pneumatic cylinders 131 would then be activated to a specific pressure to create a brace force, and the test would continue to run until the twenty seconds ended. Pneumatic cylinders 131 were inflated to five different pressures: 40, 50, 60, 70, and 80 PSI. Each pressure setting was tested ten times. Force and moment values were recorded for all runs.

Brace force F_{b} for each run was found by taking the user's bodyweight (490 N), which can be found by readings of a left load cell force F_{LLC} and of a right load cell force F_{RLC} before brace activation, and subtracting out the sum of the two load cell readings after brace activation:

F_{b}=BW−F_{LLC}−F_{RLC}

How well dynamic ankle orthosis system 100 transferred offloading forces to the limb, or Efficiency, was calculated using the brace force F_{b} of dynamic ankle orthosis system 100 and total cylinder force provided by pneumatic cylinders 131.

TABLE 2
Mean brace force for each pressure setting

| | | % BW | |
|---|---|---|---|
| PSI | Fb | Offloading | Efficiency |
| 40 | 54.7 ± 7.3 | 11.3% | 83.4% |
| 50 | 75.1 ± 3.7 | 15.5% | 86.1% |
| 60 | 106.9 ± 5.9 | 22.0% | 95.4% |
| 70 | 123.5 ± 5.9 | 25.4% | 91.4% |
| 80 | 148.0 ± 7.9 | 30.5% | 95.5% |

As a percentage of bodyweight BW, the amount of force dynamic ankle orthosis system 100 relieved ranged from 11.3% at 40 PSI to 30.5% at 80 PSI. It is currently unknown what amount of force relief at the ankle is significant, but through conversation with two orthotists, two general goals were set for dynamic ankle orthosis system 100. First, dynamic ankle orthosis system 100 should provide axial unloading without compromising circulation or soft tissue integrity. The medical literature shows that during noninvasive ankle distraction no nerve damage was seen when tested up to 225 N for 1 hour. (See, Dowdy et al.) This is not directly applicable as this experiment was performed on an unloaded ankle and the ankle would be under load with the dynamic brace design, but it still gave a reference of what was safe without causing nerve damage in an unloaded ankle. Second, a goal of approximately 25% of bodyweight of force relief at the braced ankle was set as a mark of potential clinical efficacy.

This test shows the ability of dynamic ankle orthosis system 100 to provide offloading of the ankle. Dynamic ankle orthosis system 100 provided up to 148 N of brace force F_{b} to the user which amounted to 30.5% of body weight. Dynamic ankle orthosis system 100 was also able to transfer cylinder force output to brace force F_{b} at an 83-95% effectiveness rate. Dynamic ankle orthosis system 100 therefore accomplished both goals set by the orthotists.

### Experiment 3: Mobility

The second variable of interest is the effect of the DAO on the ankle's resistance to motion. This experiment was separated into two treatment groups: without a brace and with a brace inflated to various levels of cylinder pressure. For all tests, a Biodex unit was set to passively drive the ankle in plantar- and dorsi-flexion. The user sat in a chair and a limb support pad was placed under the thigh so that the lower leg approached the machine parallel to the floor. The right foot was placed on a foot plate, and securely attached thereto to limit motion in the foot. The foot plate was then adjusted so that the dynamometer of the Biodex unit was aligned with the lateral malleolus of the right ankle. This was done so that the Biodex unit rotated about the rotational axis (RA) of the user's ankle.

The Biodex unit was set to rotate passively, which means that the machine would drive motion between two set points at a set speed. The two points were set prior to each run by the user. The foot was initially positioned at a neutral angle, perpendicular to the leg, and then the user set maximum plantarflexion and dorsiflexion angles. The machine rotated back and forth between these maximum angles for a set number of cycles and measured the moments experienced by the machine while moving the foot to these points. The data sets attained from the tests were the angular positions and the corresponding moment values. All tests were run at an angular velocity of 30 degrees/sec. For all tests the ankle was rotated to at least 10 degrees of dorsiflexion and 20 degrees of plantarflexion. The polarity of the measured moment values depends on the direction of the resistive moment. Dorsiflexor moments (directed towards the top of the foot) are measured as negative resistive moments, and plantarflexor moments (directed towards the bottom of the foot) are measured as positive resistive moments). So as the foot is passively moved into dorsiflexion, a resistive plantarflexor moment is generated by the soft tissue of the calf and ankle. First, the foot was placed in the Biodex machine without dynamic ankle orthosis system 100 donned. The straps were securely tightened at the foot to ensure that rotation occurred at the ankle joint. Three tests were run, each for thirty seconds with the system angular velocity set to 30 deg/sec. The mean moments and standard deviations were then calculated at 5 degree increments from −10 degrees to 20 degrees. Moment values for the plate by itself were subtracted out so the reported moment values accurately represent what is added to the Biodex system. These values provided a baseline to compare the results of the different bracing conditions to that of the foot alone.

Five different bracing conditions were tested at the neutral position. The user sat in the chair as above, with dynamic ankle orthosis system 100 donned and the foot secured to the foot plate. Each test was run for 30 seconds and contained between 7 and 10 full revolutions of the foot, moving from maximum dorsiflexion to maximum plantarflexion and back. Tests were run with the pneumatic cylinders filled to 0, 40, 50, 60, 70, and 80 PSI values. Each pressure setting was run three times and the recorded output values were the moments and the corresponding angular position values. Moment values at −10, −5, 0, 5, 10, and 15 degrees were used for analysis. 0 degrees represents when the foot is positioned at a 90 degree angle to the lower leg. Negative angle values indicate dorsiflexion and positive values plantarflexion.

Following in Table 3 are the mean moment values for the non-braced and braced conditions. At each five degree increment in both directions mean moment was compared amongst all different bracing conditions.

TABLE 3
Non-braced and braced moment values at 5 degree increments
ANKLE MOMENT (NM)

| | Ankle Angular Position (Degrees) | | | | | | |
|---|---|---|---|---|---|---|---|
| | Dorsiflexion (−) | | Neutral | Plantar Flexion (+) | | | |
| | −10° | −5° | 0° | 5° | 10° | 15° | 20° |
| Native Ankle | 6.0 | 4.5 | 3.7 | 3.1 | 2.4 | 1.9 | 1.5 |
| DAO 0 PSI | 4.2 | 3.3 | 2.6 | 1.8 | 1.2 | 0.7 | 0.1 |
| DAO 40 PSI | 2.6 | 1.8 | 0.9 | 0.4 | 0.0 | −0.5 | −1.1 |
| DAO 50 PSI | 2.6 | 1.8 | 1.1 | 0.4 | 0.0 | −0.7 | −1.8 |
| DAO 60 PSI | 2.6 | 1.6 | 0.8 | 0.3 | −0.3 | −0.9 | −1.9 |
| DAO 70 PSI | 2.6 | 1.6 | 0.7 | 0.0 | −0.5 | −1.2 | −2.3 |
| DAO 80 PSI | 1.8 | 0.8 | 0.0 | −0.7 | −1.4 | −2.2 | −4.2 |

The measured moment values for the native ankle are supported by literature (Kay 2009). Across the range of motion (10 degrees dorsiflexion to 20 degrees plantarflexion), the native ankle experienced between 1.5 and 6.0 Nm (4.5 Nm difference) of resistive moment. With dynamic ankle orthosis system 100 donned and cylinders depressurized, between 4.2 and 0.1 ft-lbs (4.1 Nm moment difference) of resistive moment was measured during the motion. With dynamic ankle orthosis system 100 donned and inflated to 50 PSI, between 2 and −1.8 Nm (4.4 ft-lbs moment difference) of resistive moment was measured during the motion. With dynamic ankle orthosis system 100 donned and inflated to 80 PSI, between 1.8 and −4.2 ft-lbs (6 Nm moment difference) of resistive moment was measured during the motion. The increased moment at 20 degrees plantarflexion was likely due to the ball joints of dynamic ankle orthosis system 100 hitting their limit (i.e., the ball joints were constructed to permit up to 20 degrees of motion). In all pressurized bracing conditions, the absolute resistive moment was reduced compared to the native ankle. This shift in values towards the negative was likely due to the brace force vector creating off-axis loads relative to the point of rotation of the Biodex foot plate. In other words, dynamic ankle orthosis system 100 may have been applying an external dorsiflexor moment, which manifested as a shift in the measured moment values towards the negative. However, the resistive moment difference taken at the two extreme ends of motion for each condition was not significantly affected by the brace wear.

The greatest moment differences were found in the 70 PSI and 80 PSI bracing conditions, where the moment difference was 5.2 Nm and 6 Nm, respectively. Compared to the native ankle at 4.5 Nm moment difference, the increase in resistive ankle moment is negligible, and it can be concluded that the presence of dynamic ankle orthosis system 100 does not introduce additional resistance to natural ankle motion.

The present subject matter can be embodied in other forms without departure from the spirit and essential characteristics thereof. The embodiments described therefore are to be considered in all respects as illustrative and not restrictive. Although the present subject matter has been described in terms of certain preferred embodiments, other embodiments that are apparent to those of ordinary skill in the art are also within the scope of the present subject matter.

### REFERENCES

Thomas R H, Daniels T R. Ankle Arthritis. The Journal of Bone and Joint Surgery, 2003 Volume 85, 923-36.
Kitaoka et al. The Effect of Custom-Made Braces for the Ankle and Hindfoot on Ankle and Foot Kinematics and Ground Reaction Forces. Phys Med Rehabil. Volume 87, 130-5.
Kay et al. Moderate-duration static stretch reduces active and passive plantar flexor moment but not Achilles tendon stiffness or active muscle length. J Appl Physiol 106: 1249-1256, 2009.

1. A dynamic ankle orthosis system comprising:
a calf sleeve configured to be secured about a leg of a user;
a foot plate configured to be secured about a foot of the user; and
a distractive force mechanism connected between the calf sleeve and the foot plate, wherein the distractive force mechanism is configured to generate a force between the foot plate and the calf sleeve acting bidirectionally across an ankle of the user to substantially offload bodyweight of the user passing through the foot, ankle, and leg.

2. The dynamic ankle orthosis system of claim 1, wherein the calf sleeve comprises one or more anterior engagement elements configured to engage a shin of the user and one or more posterior engagement elements configured to engage a calf of the user.

3. The dynamic ankle orthosis system of claim 2, wherein the one or more anterior engagement elements comprises one or more rigid or semi-rigid portions configured to mate with a natural anatomical profile of a flat portion of a tibial shaft of the user.

4. The dynamic ankle orthosis system of claim 2, wherein the calf sleeve comprises a calf ring fixed to a portion of the one or more anterior engagement elements; and
wherein the distractive force mechanism is connected to the calf sleeve at the calf ring.

5. The dynamic ankle orthosis system of claim 2, wherein the one or more posterior engagements elements comprise one or more elements selected from the group consisting of a textile mesh, a bladder, and padding.

6. The dynamic ankle orthosis system of claim 2, wherein the one or more anterior engagement elements are coupled to the one or more posterior engagement elements by a cable tensioning system.

7. The dynamic ankle orthosis system of claim 1, wherein the distractive force mechanism is connected to the foot plate at a position that substantially corresponds with a location of a rotational axis of the ankle of the user.

8. The dynamic ankle orthosis system of claim 7, wherein the distractive force mechanism is connected to an adapter plate that is connected to the foot plate, wherein a position of the adapter plate relative to the foot plate is adjustable.

9. The dynamic ankle orthosis system of claim 1, wherein the distractive force mechanism comprises one or more pneumatic cylinders coupled between the calf sleeve and the foot plate.

10. The dynamic ankle orthosis system of claim 9, wherein the one or more pneumatic cylinders are connected to a pressure control assembly that is operable to maintain a selected pressure within the one or more pneumatic cylinders.

11. The dynamic ankle orthosis system of claim 9, wherein the one or more pneumatic cylinders comprises first and second pneumatic cylinders, wherein bottom ends of each of the first and second pneumatic cylinders are connected in a substantially closed system so that extension of the first pneumatic cylinder causes shortening of the second pneumatic cylinder, thereby allowing unconstrained inversion or eversion ankle motion under an active loading state.

12. The dynamic ankle orthosis system of claim 9, wherein the one or more pneumatic cylinders comprises first and second pneumatic cylinders, wherein a first air tube is connected to a top of the first pneumatic cylinder, wherein a second air tube can be connected to a bottom of the second pneumatic cylinder, wherein the distractive force mechanism is configured to be loaded but neither of the first or second pneumatic cylinders is changeable in length.

13. The dynamic ankle orthosis system of claim 1, wherein the distractive force mechanism comprises:
one or more constant force springs;
one or more rods secured between the calf sleeve and the foot plate; and
one or more tension cables connected between an end of one of the one or more constant force springs and a corresponding one of the one or more rods;
wherein the one or more rods are configured to be displaced towards the foot plate when tension is applied to a corresponding one of the one or more tension cables.

14. The dynamic ankle orthosis system of claim 1, wherein the distractive force mechanism is connected to the calf sleeve by one or more tie rod ends.

15. The dynamic ankle orthosis system of claim 1, wherein connections of the distractive force mechanism to the foot plate are configured to be substantially aligned with a rotational axis of the ankle of the user.

16. The dynamic ankle orthosis system of claim 15, wherein the connections of the distractive force mechanism to the foot plate comprise ball joints.

17. A method for offloading at least a portion of a user's bodyweight at an ankle of the user, the method comprising:
securing a calf sleeve about a leg of the user;
securing a foot plate about a foot of the user;
connecting a distractive force mechanism between the calf sleeve and the foot plate; and
generating a force by the distractive force mechanism between the foot plate and the calf sleeve acting bidirectionally across an ankle of the user to substantially offload bodyweight of the user passing through the foot, ankle, and leg.

18. The method of claim 17, wherein securing a calf sleeve about a leg of the user comprises:
engaging one or more anterior engagement elements of the calf sleeve with a shin of the user; and
engaging one or more posterior engagement elements of the calf sleeve with a calf of the user.

19. The method of claim 18, wherein engaging one or more anterior engagement elements of the calf sleeve with a shin of the user comprises mating one or more rigid or semi-rigid portions of the calf sleeve with a natural anatomical profile of a flat portion of a tibial shaft of the user.

20. The method of claim 18, wherein the calf sleeve comprises a calf ring fixed to a portion of the one or more anterior engagement elements; and
wherein connecting a distractive force mechanism between the calf sleeve and the foot plate comprises connecting the distractive force mechanism to the calf sleeve at the calf ring.

21. The method of claim 18, wherein the one or more posterior engagement elements comprise one or more elements selected from the group consisting of a textile mesh, a bladder, and padding.

22. The method of claim 18, comprising coupling the one or more anterior engagement elements to the one or more posterior engagement elements by a cable tensioning system.

23. The method of claim 17, wherein connecting the distractive force mechanism between the calf sleeve and the foot plate comprises connecting the distractive force mechanism to the foot plate at a position that substantially corresponds with a location of a rotational axis of the ankle of the user.

24. The method of claim 23, wherein connecting the distractive force mechanism to the foot plate comprises connecting the distractive force mechanism to an adapter plate that is connected to the foot plate, wherein a position of the adapter plate relative to the foot plate is adjustable.

25. The method of claim 17, wherein connecting the distractive force mechanism between the calf sleeve and the foot plate comprises coupling one or more pneumatic cylinders between the calf sleeve and the foot plate.

26. The method of claim 25, comprising connecting the one or more pneumatic cylinders to a pressure control assembly; and
operating the pressure control assembly to maintain a selected pressure within the one or more pneumatic cylinders.

27. The method of claim 25, wherein the one or more pneumatic cylinders comprises first and second pneumatic cylinders, wherein bottom ends of each of the first and second pneumatic cylinders are connected in a substantially closed system so that extension of the first pneumatic cylinder causes shortening of the second pneumatic cylinder, thereby allowing unconstrained inversion or eversion ankle motion under an active loading state.

28. The method of claim 25, wherein the one or more pneumatic cylinders comprises first and second pneumatic cylinders, wherein a first air tube is connected to a top of the first pneumatic cylinder, wherein a second air tube can be connected to a bottom of the second pneumatic cylinder, wherein the distractive force mechanism is configured to be loaded but neither of the first or second pneumatic cylinders is changeable in length.

29. The method of claim 17, wherein connecting the distractive force mechanism between the calf sleeve and the foot plate comprises:
coupling one or more constant force springs to the calf sleeve;
coupling one or more rods between the calf sleeve and foot plate; and
connecting one or more tension cables between an end of one of the one or more constant force springs and a corresponding one of the one or more rods;
wherein generating a force comprises displacing the one or more rods towards the foot plate when tension is applied to a corresponding one of the one or more tension cables.

30. The method of claim 17, wherein the distractive force mechanism is connected to the calf sleeve by one or more tie rod ends.

31. The method of claim 17, wherein connections of the distractive force mechanism to the foot plate are substantially aligned with a rotational axis of the ankle of the user.

32. The method of claim 31, wherein the connections of the distractive force mechanism to the foot plate comprise ball joints.
